(19) United States
(12) Patent Application Publication
(10) Pub. No.: US 2022/0288149 A1
(43) Pub. Date: Sep. 15, 2022

(54) SENSITIZATION CREAM COMPRISING L-ARGININE AND L-CITRULLINE AND THERAPEUTIC USES THEREOF

(71) Applicant: INNOVUS PHARMACEUTICALS, INC., San Diego, CA (US)

(72) Inventor: Bassam DAMAJ, San Diego, CA (US)

(21) Appl. No.: 17/635,248

(22) PCT Filed: Aug. 14, 2020
(86) PCT No.: PCT/US2020/046398

§ 371 Date: Feb. 14, 2022

Related U.S. Application Data
(60) Provisional application No. 62/886,861, filed on Aug. 14, 2019.

(51) Int. Cl. A61K 36/736; A61K 8/44; A61K 8/92; A61Q 19/00; A61K 8/06; A61K 36/54; A61K 36/23; A61K 31/198; A61P 25/02; A61K 9/00; A61K 9/06; A61K 47/26; A61K 47/10; A61K 47/18; A61K 47/32

(52) U.S. Cl. CPC A61K 36/736; A61K 8/44; A61K 8/922; A61Q 19/005; A61K 8/062; A61K 36/54; A61K 36/23; A61K 31/198; A61P 25/02; A61K 9/0014; A61K 9/06; A61K 47/26; A61K 47/10; A61K 47/18; A61K 47/183; A61K 47/32

(57) ABSTRACT

Topical compositions comprising L-arginine and L-citrulline are described. Methods and uses are also provided that include the administration of an effective amount of the topical compositions. The topical compositions are useful for increasing sensitization of the skin and/or for increasing blood flow to the skin.

### BACKGROUND

### Field

The present invention generally relates to a method and topical composition for improving blood flow to the skin and improving sensitivity of the skin. More specifically, the present invention relates to a topical formulation for increasing sensitization wherein the formulation comprises L-arginine and L-citrulline.

### Description of the Related Art

The penis is a highly sensitive organ that contains thousands of nerve ending. In order for sexual arousal to occur, these nerve endings need to be stimulated. When a loss of sensation occurs, it can be more difficult for men to become aroused, reach orgasm, and enjoy sexual activity. This loss of sensitivity can lead to an inability to enter relationships, can cause strain in relationships, reduce a partner's sexual satisfaction, and can cause psychological damage to sufferers.

A number of factors can lead to reduced penis sensitivity. Poor circulation attributable to poor cardiovascular health, or neuropathy, including peripheral neuropathy caused by systemic disorders such as diabetes (also known as diabetic neuropathy) or by hyperglycemia-induced glycation, the effect of certain medications, or traumatic injury. Other factors that can lead to reduced penis sensitivity include lack of exercise, and obesity. Drug and alcohol use, smoking, use of prescription medication such as anti-depressants, and aging are also thought to contribute to a loss of penile sensation. Additionally, circumcision may also impair penile sensation due to the removal of highly sensitive foreskin tissue.

Efforts to improve penile sensitivity have been directed toward optimizing other aspects of health, such as reducing body weight and quitting smoking. For circumcised men, foreskin restoration options may be available, however such procedures have mixed results and unwanted side effects. In particular, surgical restoration procedures may be highly risky and be prohibitively expensive for most men. Topical creams are also available for increasing penis sensitivity due to the ease of application and low cost compared with other treatment options. Examples of sensitization creams are disclosed in U.S. Pat. Nos. 9,821,021 and 9,833,488.

Improving blood flow to the penis can also enhance its function, improve symptoms of erectile dysfunction and enhance sexual arousal. U.S. Pat. No. 5,439,938 discloses a method for treating male comprising a compound that generates nitric oxide in an amount sufficient to initiate penile erection. Also, infusion of L-arginine, a precursor to nitric oxide in the body, promotes vasodilation and blood flow (See Morikawa et al, Stroke 1994; 25:429-435). Topical delivery of arginine to produce enhanced blood flow in the penis is disclosed in U.S. Pat. No. 7,914,814.

Diabetic neuropathies are a family of nerve disorders caused by diabetes. People with diabetes can, over time, develop nerve damage throughout the body, and nerve problems can occur in every organ system, including the digestive tract, heart, and sex organs. About 60 to 70 percent of people with diabetes have some form of neuropathy, and the neuropathy can cause loss of sensitivity in the feet and hands and can result in changes in sexual response, such an inability to have erections or reach sexual climax. Increasing sensitivity of the remaining nerves of neuropathy patients may result in increased sensation, ease the symptoms of neuropathy, and greatly improve a person's quality of life. An example of the treatment of neuropathy by topical application of a composition is described in U.S. Patent Publication No. 2003/0157185.

Thus there is a need for a method of increasing sensitivity of the penis and blood flow to the penis that requires no specialized therapy, can be used easily, conveniently, and without embarrassment, and does not involve the problems associated with prior therapeutic methods.

### SUMMARY

Some embodiments disclosed herein provide a topical composition for improving the sensitivity of the skin and improving blood flow to the skin comprising sweet almond oil, cinnamon bark oil, coriander seed oil, L-arginine, and L-citrulline in an oil-in-water emulsion.

In some embodiments, the composition comprises about 6 wt. % sweet almond oil, about 0.75 wt. % cinnamon bark oil, about 0.5 wt. % coriander seed oil, about 0.34 wt. % L-arginine, and about 0.68 wt. % L-citrulline.

In some embodiments, the mass ratio of L-arginine to L-citrulline in the composition is about 1:2.

In some embodiments, the mass ratio of sweet almond oil to cinnamon bark oil to coriander seed oil to L-arginine to L-citrulline in the composition is about 72:9:6:4:8.

In some embodiments, the composition further comprises one or more pharmaceutically acceptable excipients.

In some embodiments, the composition comprises: about 6 wt. % sweet almond oil, about 0.75 weight percent cinnamon bark oil, about 0.5 wt. % coriander seed oil, about 0.34 wt. % L-arginine, about 0.68 wt. % L-citrulline, about 5.0 wt. % of a 70 percent by weight sorbitol solution in water, about 4.0 wt. % of a 70 percent by weight isopropyl alcohol solution in water, about 20.0 wt. % propylene glycol, about 1.0 wt. % butylated hydroxytoluene, about 1.0 wt. % triethanolamine, about 1.0 wt. % benzyl alcohol, about 1.0 wt. % benzyl benzoate, about 3.5 wt. % PEG 40-hydrogenated castor oil, about 1.3 wt. % acrylate/C₁₀₋₃₀ alkyl acrylate crosspolymer, about 0.1 wt. % ethylenediaminetetraacetic acid disodium salt, and about 53.9 wt. % water.

Other embodiments disclosed herein include a method of improving the sensitivity of the skin or improving blood flow to the skin comprising applying to the skin an effective amount of a topical composition comprising sweet almond oil, cinnamon bark oil, coriander seed oil, L-arginine, and L-citrulline in an oil-in-water emulsion. In some embodiments, the method disclosed herein improves the sensitivity of the skin. In some embodiments, the method disclosed herein improves the blood flow to the skin. In some embodiments, the method disclosed herein improves and or provides at least partial relief for one or more symptom of neuropathy. In some embodiments, the method disclosed herein improves and or provides at least partial relief for one or more symptom of diabetic neuropathy. In some embodiments, the method disclosed herein improves the sensitivity of the skin and the blood flow to the skin. In some embodiments, the method disclosed herein improves the sensitivity of the skin and the symptoms of diabetic neuropathy. In some embodiments, the method disclosed herein improves the blood flow to the skin and the symptoms of diabetic neuropathy. In some embodiments, the method disclosed herein improves the sensitivity of the skin, the blood flow to the skin, and the symptoms of diabetic neuropathy.

In some embodiments, the composition is applied to the skin of the human penis. In some embodiments penis is circumcised. In some embodiments, the penis is uncircumcised. In some embodiments, a dose of approximately 0.5 mL, 0.6 mL, 0.7 mL, 0.8 mL, 0.9 mL, 1.0 mL, 1.1 mL, 1.2 mL, 1.3 mL, 1.4 mL, 1.5 mL, 2.0 mL, 2.5 mL, 3.0 mL, 3.5 mL, 4.0 mL, 4.5 mL, 5.0 mL or more of the composition are applied topically to the skin one or, more preferably, two to three times daily for a period of one week or, more preferably, two to three weeks to have an initial loading dose of the composition, followed by one to two applications daily or as needed.

### BRIEF DESCRIPTION OF THE DRAWINGS

FIG. 1 describes the effect of the formulation disclosed herein on blood flow in the tails of mice. DiabaSens is a trade name that denotes a commercial embodiment of the formulation of the invention.

FIG. 2 describes the effect of the formulation disclosed herein on topical pain in mice after 14 days of treatment in the Tail Flick Heat Model.

FIG. 3 describes the effect of the formulation disclosed herein on numbness reduction of hands and feet in human subjects as measured by numbness reduction score.

FIG. 4 describes the effect of the formulation disclosed herein on pain reduction of hands and feet in human subjects as measured by pain reduction score.

FIG. 5 describes the effect of the formulation disclosed herein on in sensation increase of hands and feet in human subjects as measured by sensation increase score.

FIG. 6 describes the effect of the formulation disclosed herein on swelling reduction of hands and feet in human subjects as measured by swelling reduction score.

FIG. 7 describes the effect of the formulation disclosed herein on tingling reduction of hands and feet in human subjects as measured by tingling reduction score.

FIG. 8 describes the effect of the formulation disclosed herein on balance increase in human subjects as measured by balance increase score.

### DETAILED DESCRIPTION OF THE PREFERRED EMBODIMENT

In some embodiments, a topical composition is provided comprising sweet almond oil, cinnamon bark oil, coriander seed oil, L-arginine, and L-citrulline. In some embodiments, the topical composition is formulated as an oil-in water emulsion. The topical compositions may be formulated as, for example, a gel; lotion, cream, mousse, aerosol, ointment, or lubricants, etc., so long as when the composition is applied, especially to the genitalia, the formulation will substantially stay in place. For example, there will not be substantial run-off for a sufficient time after application, to permit an individual to spread the composition over a relevant portion of the penis, preferably, over the glans of the penis.

In some embodiments, the topical compositions comprises sweet almond oil in any amount up to its solubility limit. In some embodiments, the amount of sweet almond oil in the topical composition may be, for example, at least 1.0 wt. %, at least 2.0 wt. %, at least 3.0 wt. %, at least 4.0 wt. %, at least 5.0 wt. %, at least 6.0 wt. %, at least 7.0 wt. %, at least 8.0 wt. % at least 9.0 wt. %, or at least 10.0 wt. %, or within a range defined by any two of the aforementioned concentrations. Approximately 1.0 wt. %, 1.5 wt. %, 2.0 wt. %, 2.5 wt. %, 3.0 wt. %, 3.5 wt. %, 4.0 wt. %, 4.5 wt. %, 5.0% wt. %, 5.5 wt. %, 6.0 wt. %, 6.5 wt. %, 7.0 wt. %, 7.5 wt. %, 8.0 wt. %, 8.5 wt. %, 9.0 wt. %, 9.5 wt. %, and 10.0 wt. % sweet almond oil present in the topical composition are contemplated within the scope of the invention.

In some embodiments, the topical compositions comprises cinnamon bark oil in any amount up to its solubility limit. In some embodiments, the amount of cinnamon bark oil in the topical composition may be, for example, at least 0.1 wt. %, at least 0.2 wt. %, at least 0.3 wt. %, at least 0.4 wt. %, at least 0.5 wt. %, at least 0.75 wt. %, at least 1.0 wt. %, at least 1.25 wt. %, at least 1.5 wt. %, at least 1.75 wt. %, at least 2.0 wt. %, or within a range defined by any two of the aforementioned concentrations. Approximately 0.1 wt. %, 0.2 wt. %, 0.3 wt. %, 0.4 wt. %, 0.5 wt. %, 0.55 wt. %, 0.60 wt. %, 0.75 wt. %, 0.80% wt. %, 0.85 wt. %, 0.90 wt. %, 0.95 wt. %, 1.0 wt. %, 1.05 wt. %, 1.10 wt. %, 1.15 wt. %, 1.20 wt. %, 1.25 wt. %, 1.50 wt. %, 1.75 wt. %, and 2.0 wt. % cinnamon bark oil present in the topical composition are contemplated within the scope of the invention.

In some embodiments, the topical compositions comprises coriander seed oil in any amount up to its solubility limit. In some embodiments, the amount of coriander seed oil in the topical composition may be, for example, at least 0.1 wt. %, at least 0.2 wt. %, at least 0.3 wt. %, at least 0.4 wt. %, at least 0.5 wt. %, at least 0.75 wt. %, at least 1.0 wt. or within a range defined by any two of the aforementioned concentrations. Approximately 0.05 wt. %, 0.1 wt. %, 0.15 wt. %, 0.2 wt. %, 0.25 wt. %, 0.3 wt. %, 0.35 wt. %, 0.4 wt. %, 0.45 wt. %, 0.5 wt. %, 0.55 wt. %, 0.60 wt. %, 0.75 wt. %, 0.80% wt. %, 0.85 wt. %, 0.90 wt. %, 0.95 wt. %, and 1.0 wt. % coriander seed oil present in the topical composition are contemplated within the scope of the invention.

In some embodiments, the topical compositions comprises L-arginine in any amount up to its solubility limit. In some embodiments, the amount of L-arginine in the topical composition may be, for example, at least 0.001 wt. %, at least 0.002 wt. %, at least 0.005 wt. %, at least 0.01 wt. %, at least 0.02 wt. %, at least 0.03 wt. %, at least 0.05 wt. %, at least 0.1 wt. %, at least 0.2 wt. %, at least 0.3 wt. %, at least 0.4 wt. %, at least 0.5 wt. %, at least 1.0 wt. %, at least 2.0 wt. %, at least 3.0 wt. %, at least 4.0 wt. %, at least 5.0 wt. %, or at least 6.0 wt. % or within a range defined by any two of the aforementioned concentrations. For example, in some embodiments, the amount of L-arginine present in the topical composition will range from, for example, approximately 0.05 wt. % to approximately 5.0 wt. %, from approximately 0.2 wt. % to approximately 3.0 wt. %, from approximately 0.001 wt. % to approximately 0.01 wt. %, from approximately 0.01 wt. % to approximately 0.05 wt. %, from approximately 0.05 wt. % to approximately 0.10 wt. %, from approximately 0.10 wt. % to approximately 0.20 wt. %, from approximately 0.15 wt. % to approximately 0.25 wt. %, from approximately 0.15 wt. % to approximately 1.0 wt. %, or from approximately 0.20 wt. % to approximately 0.60 wt. %. Approximately 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.3 wt. %, 0.33 wt. %, 0.67 wt. %, 1.0 wt. %, 1.33 wt. %, 1.67 wt. %, 2.0% wt. 2.33 wt. %, 2.67 wt. %, 3.0 wt. %, 4.0 wt. %, and 5.0 wt. % L-arginine present in the topical composition are contemplated within the scope of the invention.

In some embodiments, the topical compositions provided herein may comprise L-citrulline in any amount up to its solubility limit. In some embodiments, the amount of L-citrulline in the topical composition may be, for example, at least 0.001 wt. %, at least 0.002 wt. %, at least 0.005 wt. %, at least 0.01 wt. %, at least 0.02 wt. %, at least 0.03 wt. %, at least 0.05 wt. %, at least 0.10 wt. % at least 0.20 wt. % at least 0.30 wt. % at least 0.40 wt. %, at least 0.50 wt. % at least 0.60 wt. %, at least 0.70 wt. %, at least 0.80 wt. %, at least 0.90 wt. %, at least 1.0 wt. %, at least 2.0 wt. %, at least 3.0 wt. %, at least 4.0 wt. %, at least 5.0 wt. %, at least 6.0 wt. %, at least 7.0 wt. %, at least 8.0 wt. %, or within a range defined by any two of the aforementioned concentrations. For example, in some embodiments, the amount of L-citrulline present in the topical composition ranges from, approximately 0.1 wt. % to approximately 8.0 wt. %, from approximately 0.1 wt. % to approximately 6.0 wt. %, from approximately 0.001 wt. % to approximately 0.01 wt. %, from approximately 0.01 wt. % to approximately 0.05 wt. %, from approximately 0.1 wt. % to approximately 0.20 wt. %, from approximately 0.25 wt. % to approximately 0.45 wt. %, from approximately 0.25 wt. % to approximately 2 wt. %, from approximately 0.30 wt. % to approximately 0.40 wt. %, or from approximately 0.40 wt. % to approximately 0.80 wt. %. Approximately 0.1 wt. %, 0.2 wt. %, 0.3 wt. %, 0.33 wt. %, 0.35 wt. %, 0.40 wt. %, 0.50 wt. %, 0.67 wt. %, 1.0 wt. %, 1.33 wt. %, 1.67 wt. %, 2.0% wt. 2.33 wt. %, 2.67 wt. %, 3.0 wt. %, 4.0 wt. %, 5.0 wt. %, 6.0 wt. %, 7.0 wt. %, and 8.0 wt. % L-citrulline present in the topical composition are contemplated within the scope of the invention.

In some embodiments, the topical compositions comprises sorbitol solution USP 70% in any amount up to its solubility limit. In some embodiments, the amount of sorbitol solution USP 70% in the topical composition may be, for example, at least 1.0 wt. %, at least 2.0 wt. %, at least 3.0 wt. %, at least 4.0 wt. %, at least 5.0 wt. %, at least 6.0 wt. %, at least 7.0 wt. %, at least 8.0 wt. %, at least 9.0 wt. %, at least 10.0 wt. %, or within a range defined by any two of the aforementioned concentrations. Approximately 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 7.5% wt. %, 8.0 wt. %, 8.5 wt. %, 9.0 wt. %, 9.5 wt. %, and 10.0 wt. % sorbitol solution USP 70% present in the topical composition are contemplated within the scope of the invention.

In some embodiments, the topical compositions comprises isopropyl alcohol USP 70% in any amount up to its solubility limit. In some embodiments, the amount of isopropyl alcohol USP 70% in the topical composition may be, for example, at least 1.0 wt. %, at least 2.0 wt. %, at least 3.0 wt. %, at least 4.0 wt. %, at least 5.0 wt. %, at least 6.0 wt. %, at least 7.0 wt. %, at least 8.0 wt. %, at least 9.0 wt. %, at least 10.0 wt. %, or within a range defined by any two of the aforementioned concentrations. Approximately 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 7.5% wt. %, 8.0 wt. %, 8.5 wt. %, 9.0 wt. %, 9.5 wt. %, and 10.0 wt. % isopropyl alcohol USP 70% present in the topical composition are contemplated within the scope of the invention.

In some embodiments, the topical compositions comprises propylene glycol in any amount up to its solubility limit. In some embodiments, the amount of propylene glycol in the topical composition may be, for example, at least 10 wt. %, at least 15 wt. %, at least 20 wt. %, at least 25 wt. %, at least 30 wt. %, or within a range defined by any two of the aforementioned concentrations. Approximately 15.0 wt. %, 16.0 wt. %, 17.0 wt. %, 18.0 wt. %, 19.0 wt. %, 20.0 wt. %, 21.0 wt. %, 22.0 wt. %, 23.0 wt. %, 24.0 wt. %, 25.0 wt. %, 26.0 wt. %, 27.0 wt. %, 28.0% wt. %, 29.0 wt. %, and 30.0 wt. % propylene glycol present in the topical composition are contemplated within the scope of the invention.

In some embodiments, the topical compositions comprises butylated hydroxytoluene in any amount up to its solubility limit. In some embodiments, the amount of butylated hydroxytoluene in the topical composition may be, for example, at least 0.1 wt. %, at least 0.2 wt. %, at least 0.3 wt. %, at least 0.4 wt. %, at least 0.5 wt. %, at least 0.6 wt. %, at least 0.7 wt. %, at least 0.8 wt. %, at least 0.9 wt. %, at least 1.0 wt. %, at least 1.1 wt. %, at least 1.2 wt. % at least 1.3 wt. %, at least 1.4 wt. %, at least 1.5 wt. %, or within a range defined by any two of the aforementioned concentrations. Approximately 0.05 wt. %, 0.1 wt. %, 0.15 wt. %, 0.2 wt. %, 0.25 wt. %, 0.3 wt. %, 0.35 wt. %, 0.4 wt. %, 0.45 wt. %, 0.5 wt. %, 0.55 wt. %, 0.60 wt. %, 0.75 wt. %, 0.80% wt. %, 0.85 wt. %, 0.90 wt. %, 0.95 wt. %, 1.0 wt. %, %, 1.05 wt. %, 1.10 wt. %, 1.15 wt. %, 1.20% wt. %, 1.25 wt. %, 1.30 wt. %, 1.35 wt. %, 1.40 wt. %, %, 1.45 wt. %, and 1.50 wt. % butylated hydroxytoluene present in the topical composition are contemplated within the scope of the invention.

In some embodiments, the topical compositions comprises triethanolamine in any amount up to its solubility limit. In some embodiments, the amount of triethanolamine in the topical composition may be, for example, at least 0.1 wt. %, at least 0.2 wt. %, at least 0.3 wt. %, at least 0.4 wt. %, at least 0.5 wt. %, at least 0.6 wt. %, at least 0.7 wt. %, at least 0.8 wt. %, at least 0.9 wt. %, at least 1.0 wt. %, at least 1.1 wt. %, at least 1.2 wt. % at least 1.3 wt. %, at least 1.4 wt. %, at least 1.5 wt. %, or within a range defined by any two of the aforementioned concentrations. Approximately 0.05 wt. %, 0.1 wt. %, 0.15 wt. %, 0.2 wt. %, 0.25 wt. %, 0.3 wt. %, 0.35 wt. %, 0.4 wt. %, 0.45 wt. %, 0.5 wt. %, 0.55 wt. %, 0.60 wt. %, 0.75 wt. %, 0.80% wt. %, 0.85 wt. %, 0.90 wt. %, 0.95 wt. %, 1.0 wt. %, %, 1.05 wt. %, 1.10 wt. %, 1.15 wt. %, 1.20% wt. %, 1.25 wt. %, 1.30 wt. %, 1.35 wt. %, 1.40 wt. %, %, 1.45 wt. %, and 1.50 wt. % triethanolamine present in the topical composition are contemplated within the scope of the invention.

In some embodiments, the topical compositions comprises benzyl alcohol in any amount up to its solubility limit. In some embodiments, the amount of benzyl alcohol in the topical composition may be, for example, at least 0.1 wt. %, at least 0.2 wt. %, at least 0.3 wt. %, at least 0.4 wt. %, at least 0.5 wt. %, at least 0.6 wt. %, at least 0.7 wt. %, at least 0.8 wt. %, at least 0.9 wt. %, at least 1.0 wt. %, at least 1.1 wt. %, at least 1.2 wt. % at least 1.3 wt. %, at least 1.4 wt. %, at least 1.5 wt. %, or within a range defined by any two of the aforementioned concentrations. Approximately 0.05 wt. %, 0.1 wt. %, 0.15 wt. %, 0.2 wt. %, 0.25 wt. %, 0.3 wt. %, 0.35 wt. %, 0.4 wt. %, 0.45 wt. %, 0.5 wt. %, 0.55 wt. %, 0.60 wt. %, 0.75 wt. %, 0.80% wt. %, 0.85 wt. %, 0.90 wt. %, 0.95 wt. %, 1.0 wt. %, %, 1.05 wt. %, 1.10 wt. %, 1.15 wt. %, 1.20% wt. %, 1.25 wt. %, 1.30 wt. %, 1.35 wt. %, 1.40 wt. %, %, 1.45 wt. %, and 1.50 wt. % benzyl alcohol present in the topical composition are contemplated within the scope of the invention.

In some embodiments, the topical compositions comprises benzyl benzolate in any amount up to its solubility limit. In some embodiments, the amount of benzyl benzolate in the topical composition may be, for example at least 0.1 wt. %, at least 0.2 wt. %, at least 0.3 wt. %, at least 0.4 wt. %, at least 0.5 wt. %, at least 0.6 wt. %, at least 0.7 wt. %, at least 0.8 wt. %, at least 0.9 wt. %, at least 1.0 wt. %, at least 1.1 wt. %, at least 1.2 wt. % at least 1.3 wt. %, at least 1.4 wt. %, at least 1.5 wt. %, or within a range defined by any two of the aforementioned concentrations. Approximately 0.05 wt. %, 0.1 wt. %, 0.15 wt. %, 0.2 wt. %, 0.25 wt. %, 0.3 wt. %, 0.35 wt. %, 0.4 wt. %, 0.45 wt. %, 0.5 wt. %, 0.55 wt. %, 0.60 wt. %, 0.75 wt. %, 0.80% wt. %, 0.85 wt. %, 0.90 wt. %, 0.95 wt. %, 1.0 wt. %, %, 1.05 wt. %, 1.10 wt. %, 1.15 wt. %, 1.20% wt. %, 1.25 wt. %, 1.30 wt. %, 1.35 wt. %, 1.40 wt. %, %, 1.45 wt. %, and 1.50 wt. % benzyl benzolate present in the topical composition are contemplated within the scope of the invention.

In some embodiments, the topical compositions comprises PEG 40-hydrogenated castor oil in any amount up to its solubility limit. In some embodiments, the amount of PEG 40-hydrogenated castor oil in the topical composition may be, for example, at least 1.5 wt. %, at least 2.0 wt. %, at least 2.5 wt. %, at least 3.0 wt. %, at least 3.5 wt. %, at least 4.0 wt. %, at least 4.5 wt. %, at least 5.0 wt. %, at least 5.5 wt. %, or within a range defined by any two of the aforementioned concentrations. Approximately 1.5 wt. %, 2.0 wt. %, 2.5 wt. %, 3.0 wt. %, 3.5 wt. %, 4.0 wt. %, 4.5 wt. %, 5.0 wt. %, and 5.5 wt. % PEG 40-hydrogenated castor oil present in the topical composition are contemplated within the scope of the invention.

In some embodiments, the topical compositions comprises acrylate/C₁₀₋₃₀ alkyl acrylate crosspolymer in any amount up to its solubility limit. In some embodiments, the amount of acrylate/C₁₀₋₃₀ alkyl acrylate crosspolymer in the topical composition may be, for example, at least 0.5 wt. %, at least 1.0 wt. %, at least 1.5 wt. %, at least 2.0 wt. %, or within a range defined by any two of the aforementioned concentrations. Approximately 0.5 wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, and 2.0 wt. % acrylate/C₁₀₋₃₀ alkyl acrylate crosspolymer present in the topical composition are contemplated within the scope of the invention.

In some embodiments, the topical compositions comprises disodium EDTA in any amount up to its solubility limit. In some embodiments, the amount of disodium EDTA in the topical composition may be, for example, at least 0.01 wt. %, at least 0.05 wt. %, at least 0.10 wt. %, at least 0.15 wt. %, at least 0.20 wt. %, or within a range defined by any two of the aforementioned concentrations. Approximately 0.05 wt. %, 0.06 wt. %, 0.07 wt. %, 0.08 wt. %, 0.09 wt. %, 0.10 wt. %, 0.11 wt. %, 0.12 wt. %, 0.13 wt. %, 0.14 wt. %, and 0.15 wt. % disodium EDTA present in the topical composition are contemplated within the scope of the invention.

In some embodiments, the topical compositions comprises water in a quantity sufficient such that the all of the components of the topical composition add to 100 percent.

In some embodiments the topical composition may comprise a 1:2 mass ratio of L-arginine to L-citrulline

In some embodiments the topical composition may comprise a mass ratio of L-arginine to L-citrulline of 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, or greater than 1:3.

In some embodiments the topical composition may comprise a mass ratio of 72:9:6:4:8 of sweet almond oil, cinnamon bark oil, coriander seed oil, L-arginine, and L-citrulline, respectively.

In some embodiments the topical composition may comprise a mass ratio of 1200:150:100:37:70 of sweet almond oil, cinnamon bark oil, coriander seed oil, L-arginine, and L-citrulline, respectively.

In some embodiments, the topical composition provided herein may comprise a pharmaceutical carrier, diluent, co-solvent, emulsifier, penetration enhancer, preservative, emollient, or a combination thereof. Acceptable carriers or diluents for therapeutic use are well-known in the pharmaceutical art, and are described, for example, in Remington's Pharmaceutical Sciences, 18th Ed., Mack Publishing Co., Easton, Pa. (1990), which is incorporated herein by reference in its entirety.

Preservatives, stabilizers, dyes, fragrances, and the like may be provided in the topical composition. For example, sodium benzoate, ascorbic acid, benzyl benzoate, and esters of p-hydroxybenzoic acid may be added as preservatives. In addition, antioxidants and suspending agents may be used. In one or more of the comtemplated embodiments, alcohols, esters, sulfated aliphatic alcohols, and the like may be used as surface active agents; cellulose acetate phthalate as a derivative of a carbohydrate such as cellulose or sugar, or methylacetate-methacrylate copolymer as a derivative of polyvinyl may be used as suspension agents; and plasticizers such as ester phthalates and the like may be used as suspension agents.

In certain embodiments, the topical composition may comprise sorbitol, isopropyl alcohol, propylene glycol, butylated hydroxytoluene, triethanolamine, benzyl alcohol, benzyl benzolate, PEG 40-hydrogenated castor oil, acrylate/C₁₀₋₃₀ alkyl acrylate crosspolymer, disodium EDTA, or water; or a combination thereof.

In other embodiments, provided herein is a method for improving the sensitivity of the skin comprising applying to the skin an effective amount of a topical composition comprising sweet almond oil, cinnamon bark oil, coriander seed oil, L-arginine, and L-citrulline.

In some embodiments, provided herein is a method for improving blood flow to the skin comprising applying to the skin an effective amount of a topical composition comprising sweet almond oil, cinnamon bark oil, coriander seed oil, L-arginine, and L-citrulline.

In some embodiments, provided herein is a method for improving the symptoms of neuropathy comprising applying to the skin an effective amount of a topical composition comprising sweet almond oil, cinnamon bark oil, coriander seed oil, L-arginine, and L-citrulline. In some embodiments, provided herein is a method for providing at least partial relief of at least one of the symptom of neuropathy comprising applying to the skin an effective amount of a topical composition comprising sweet almond oil, cinnamon bark oil, coriander seed oil, L-arginine, and L-citrulline. In a preferred embodiment, the neuropathy is diabetic neuropathy.

In some embodiments, provided herein is a method for improving blood flow to the skin and improving sensitivity of the skin in a subject comprising applying to the skin an effective amount of a topical composition comprising sweet almond oil, cinnamon bark oil, coriander seed oil, L-arginine, and L-citrulline.

In some embodiments, provided herein is a method for improving blood flow to the skin and improving the symptoms of diabetic neuropathy in a subject comprising applying to the skin an effective amount of a topical composition comprising sweet almond oil, cinnamon bark oil, coriander seed oil, L-arginine, and L-citrulline.

In some embodiments, provided herein is a method for reducing numbness in a subject comprising applying to the skin an effective amount of a topical composition comprising sweet almond oil, cinnamon bark oil, coriander seed oil, L-arginine, and L-citrulline. In some embodiments, the topical composition comprising sweet almond oil, cinnamon bark oil, coriander seed oil, L-arginine, and L-citrulline may be applied to the hands and feet to reduce numbness in the hands and feet.

In some embodiments, provided herein is a method for reducing pain in a subject comprising applying to the skin an effective amount of a topical composition comprising sweet almond oil, cinnamon bark oil, coriander seed oil, L-arginine, and L-citrulline. In some embodiments, the topical composition comprising sweet almond oil, cinnamon bark oil, coriander seed oil, L-arginine, and L-citrulline may be applied to the hands and feet to reduce pain in the hands and feet.

In some embodiments, provided herein is a method for increasing sensation in a subject comprising applying to the skin an effective amount of a topical composition comprising sweet almond oil, cinnamon bark oil, coriander seed oil, L-arginine, and L-citrulline. In some embodiments, the topical composition comprising sweet almond oil, cinnamon bark oil, coriander seed oil, L-arginine, and L-citrulline may be applied to the hands and feet to increase sensation in the hands and feet.

In some embodiments, provided herein is a method for reducing swelling in a subject comprising applying to the skin an effective amount of a topical composition comprising sweet almond oil, cinnamon bark oil, coriander seed oil, L-arginine, and L-citrulline. In some embodiments, the topical composition comprising sweet almond oil, cinnamon bark oil, coriander seed oil, L-arginine, and L-citrulline may be applied to the hands and feet to reduce swelling in the hands and feet.

In some embodiments, provided herein is a method for reducing tingling in a subject comprising applying to the skin an effective amount of a topical composition comprising sweet almond oil, cinnamon bark oil, coriander seed oil, L-arginine, and L-citrulline. In some embodiments, the topical composition comprising sweet almond oil, cinnamon bark oil, coriander seed oil, L-arginine, and L-citrulline may be applied to the hands and feet to reduce tingling in the hands and feet.

In some embodiments, provided herein is a method for increasing balance in a subject comprising applying to the skin an effective amount of a topical composition comprising sweet almond oil, cinnamon bark oil, coriander seed oil, L-arginine, and L-citrulline. In some embodiments, the topical composition comprising sweet almond oil, cinnamon bark oil, coriander seed oil, L-arginine, and L-citrulline may be applied to the hands and/or feet.

In some embodiments, provided herein is a method for improving sensitivity of the skin and improving the symptoms of diabetic neuropathy comprising applying to the skin an effective amount of a topical composition comprising sweet almond oil, cinnamon bark oil, coriander seed oil, L-arginine, and L-citrulline.

In some embodiments, provided herein is a method for improving blood flow to the skin, improving sensitivity of the skin, and improving the symptoms of diabetic neuropathy comprising applying to the skin an effective amount of a topical composition comprising sweet almond oil, cinnamon bark oil, coriander seed oil, L-arginine, and L-citrulline.

In certain embodiments, the topical compositions provided herein are intended for topical, non-invasive, application to the genital regions, especially the penis, its entirety, or preferably just the glans of the penis. In addition, the composition may be applied to the scrotum and/or perineum. One particular advantage of the compositions provided herein is that the compositions are effective even when applied to only the glans penis.

In certain embodiments, the topical composition may be applied to the skin of the human penis in order to improve sensitivity. In some embodiments, the topical composition may be applied to the skin of the human penis to improve blood flow. In some embodiments, the topical composition may be applied to a circumcised human penis to improve sensitivity. In some embodiments, the topical composition may be applied to a circumcised human penis to blood flow. In some embodiments, the topical composition may be applied to the skin of an uncircumcised human penis to improve sensitivity. In some embodiments, the topical composition may be applied to the skin of an uncircumcised human penis to improve blood flow.

For increasing sensitivity of the penis during sexual activity, and with the preferred application on the glans penis, the amount of formulation to be applied is preferably in the range of from about 10 to about 1000, from about 50 to about 500, from about 100 to about 400, or from about 150 to about 300 mg per application, or may be within a range defined by any of two of the aforementioned amounts per application.

In some embodiments, the composition is administered 1 to 4 times per day, preferably 2 times a day. In some embodiments, the compounds will be administered for a period of continuous therapy, for example for one day, two days, three days, a week, two weeks, one month, or more, or for months or years.

In some embodiments, the compounds will be administered for a period of continuous therapy, for example for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 month, 2 months, 3 months, 4 months, 5 months, 6 months, 7 months, 8 months, 9 months, 10 months, 11 months, 12 months, 1 year, 2 years, or more. In some embodiments, the period of administration may be within a range defined by any two of the aforementioned durations.

In some embodiments, the onset of effect after application of the composition disclosed herein may be 1 minute, 1.5 minutes, 2 minutes, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, 10 minutes, 10.5 minutes, 11 minutes, 11.5 minutes, 12 minutes, 12.5 minutes, 13 minutes, 13.5 minutes, 14 minutes, 14.5 minutes, 15 minutes, 15.5 minutes, 16 minutes, 16.5 minutes, 17 minutes, 17.5 minutes, 18 minutes, 18.5 minutes, 19 minutes, 19.5 minutes, 20 minutes, 21 minutes, 22 minutes, 23 minutes, 24 minutes, 25 minutes, 26 minutes, 27 minutes, 28 minutes, 29 minutes, 30 minutes, 35 minutes, 40 minutes, 45 minutes, 50 minutes, 55 minutes, or 60 minutes. In some embodiments, the onset of effect after application may be within a range defined by any two of the aforementioned durations. For example, in some embodiments, the onset of effect may be from 1 to 30 minutes after application, from 5 to 20 minutes after application, or from 10 to 15 minutes after application.

The terms “pharmaceutically acceptable carrier” and “pharmaceutically acceptable excipient,” as used herein, include any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

The term “excipient,” as used herein, refers to an inert or relatively inert substance that is added to a pharmaceutical composition to impart certain properties to the composition including, without limitation, improved or desired bulk, consistency, stability, binding ability, lubrication, disintegrating ability, etc. A “diluent” is a type of excipient.

### Example 1

Materials used in preparing the topical sensitization cream described herein may be made by known methods or are commercially available. It is also possible to make use of variants which are themselves known to those of ordinary skill in this art, but are not mentioned in greater detail. The skilled artisan given the literature and this disclosure is well equipped to prepare the formulations of the instant application.

Representative topical formulations according to the invention are shown in Table 1 below, with the amounts for “broad”, and preferred “narrower” ranges.

TABLE 1
Representative Topical Compositions

| | Broader Range | Narrower Range |
|---|---|---|
| Component | wt. % | wt. % |
| sweet almond oil¹ | 3.0-10.0 | 5.0-7.0 |
| cinnamon bark oil² | 0.1-2.0 | 0.5-1.25 |
| coriander seed oil³ | 0.1-1.0 | 0.25-0.75 |
| L-arginine | 0.05-5.0 | 0.2-0.6 |
| L-citrulline | 0.1-8.0 | 0.4-0.8 |
| sorbitol solution USP 70% | 1.0-10.0 | 3.0-7.0 |
| isopropyl alcohol USP 70% | 1.0-10.0 | 2.0-6.0 |
| propylene glycol | 10.0-30.0 | 15.0-25.0 |
| butylated hydroxytoluene | 0.1-3.0 | 0.5-1.5 |
| triethanolamine | 0.1-3.0 | 0.5-1.5 |
| benzyl alcohol | 0.1-3.0 | 0.5-1.5 |
| benzyl benzolate | 0.1-3.0 | 0.5-1.5 |
| PEG 40-hydrogenated castor | 1.5-5.5 | 2.5-4.5 |
| oil | | |
| acrylate/C₁₀₋₃₀ alkyl acrylate | 0.5-2.0 | 1.0-1.6 |
| crosspolymer | | |
| disodium EDTA | 0.01-0.20 | 0.05-0.15 |
| water | 40.0-60.0 | 50.0-56.0 |

¹Prunus Amygdalus Dulcis oil;
²Cinnamomum Zeylanicum bark oil;
³Coriandrum Sativum seed oil

### Example 2

Materials used in preparing the topical sensitization cream described herein may be made by known methods or are commercially available. It is also possible to make use of variants which are themselves known to those of ordinary skill in this art, but are not mentioned in greater detail. The skilled artisan given the literature and this disclosure is well equipped to prepare the formulations of the instant application.

Representative topical formulations according to the invention are shown in Table 2 below, with the amounts for “broad” and “preferred alternative narrower” ranges.

TABLE 2
Representative Topical Compositions

| | Broader Range | Narrower Range |
|---|---|---|
| Component | wt. % | wt. % |
| sweet almond oil¹ | 3.0-10.0 | 5.0-7.0 |
| cinnamon bark oil² | 0.1-2.0 | 0.5-1.25 |
| coriander seed oil³ | 0.1-1.0 | 0.25-0.75 |
| L-arginine | 0.05-5.0 | 0.15-0.25 |
| L-citrulline | 0.1-8.0 | 0.25-0.45 |
| sorbitol solution USP 70% | 1.0-10.0 | 3.0-7.0 |
| isopropyl alcohol USP 70% | 1.0-10.0 | 2.0-6.0 |
| propylene glycol | 10.0-30.0 | 15.0-25.0 |
| butylated hydroxytoluene | 0.1-3.0 | 0.5-1.5 |
| triethanolamine | 0.1-3.0 | 0.5-1.5 |
| benzyl alcohol | 0.1-3.0 | 0.5-1.5 |
| benzyl benzolate | 0.1-3.0 | 0.5-1.5 |
| PEG 40-hydrogenated castor | 1.5-5.5 | 2.5-4.5 |
| oil | | |
| acrylate/C₁₀₋₃₀ alkyl acrylate | 0.5-2.0 | 1.0-1.6 |
| crosspolymer | | |
| disodium EDTA | 0.01-0.20 | 0.05-0.15 |
| water | 40.0-60.0 | 50.0-56.0 |

¹Prunus Amygdalus Dulcis oil;
²Cinnamomum Zeylanicum bark oil;
³Coriandrum Sativum seed oil

### Example 3

### Effect of Composition on Blood Flow

A total of 36 CD-1 mice (Taconic Farms) aged 6 to 8 weeks were randomized according to their weights into three groups: Mice in Group 1 were left untreated to serve as a control group; mice in Group 2 received a placebo composition; and mice in Group 3 received the treatment composition as described in Example 1 and Table 1. Mice were dosed with the treatment composition or placebo by applying 0.1 mL of the composition directly to the tail. The mice were treated three times a day for 14 days, with treatment commencing on day 1.

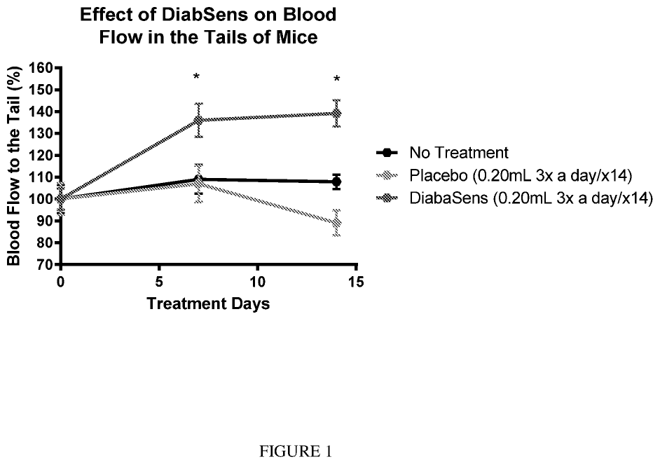

The mice were acutely anesthetized with isoflurane (approximately 2%-3.5% at 1 L/min for all measurements. All blood flow measurements were made with a MoorLab laser Doppler (Moor Instruments) perfusion monitor with an appropriately sized Doppler probe and calibrated with the “Probe Flux” standard. Blood flow was measured on days 0, 7, and 14 at a consistent site on the tail identified with a permanent marker. The Doppler probe was place just above the tail capillary but not in contact with the tail tissue. FIG. 1 shows a statistically significant increase in blood flow in the tails of mice treated with the test composition as compared with either place-treated or untreated mice.

### Example 4

### Effect of Composition on Topical Pain

A total of 30 CD-1 mice (Taconic Farms) aged 6 to 8 weeks were randomized into two groups: Mice in Group 1 were treated with a placebo composition and mice in Group 2 were treated with treatment composition of Example 1 and Table 1. Mice were dosed with the treatment composition or placebo by applying 0.1 mL of the composition directly to the tail. The mice were treated three times a day for 14 days, with treatment commencing on day 1.

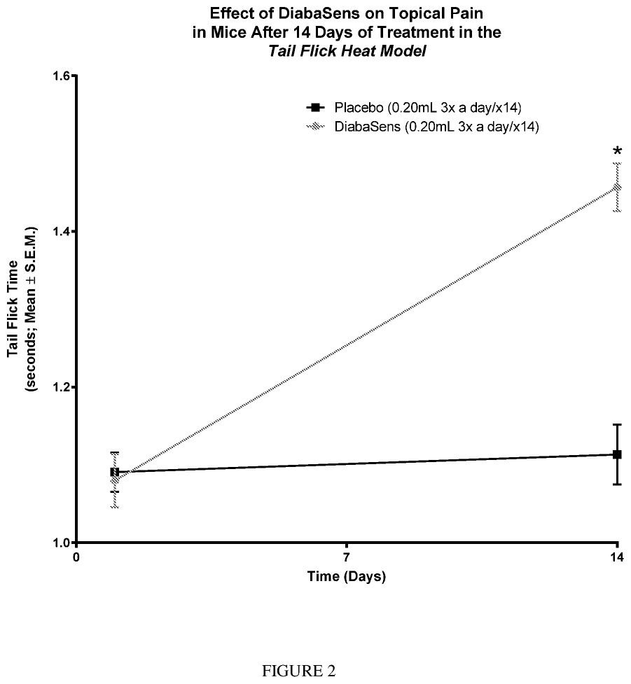

The tail flick time (i.e. the time it takes for the mouse's tail to flick after being placed on a hot plate) was measured at day 1 and day 14. FIG. 2 shows a statistically significant increase in tail flick time of mice treated with the test composition as compared with placebo-treated mice.

### Example 5

### Clinical Use Survey

Inclusion criteria for the clinical use survey were adults at least 18 years of age and currently using the topical composition of Example 2 and Table 2 disclosed herein. The parameters were subjective responses pre and post use, evaluating changes in sensitivity (pain, numbness, feeling, tingling), swelling, cutaneous manifestations (cracking), balance, speed of onset following use, and global product and quality of life assessment. Demographics and history of diabetes and/or neuropathy were recorded. The parameters were assessed using an ordinal 10-point scale where 10 was the highest scoring (extremely painful, extremely bothersome) and 1 was the lowest (none or not bothersome). Global assessments were a simple Yes or No response. The topical composition was applied as needed to either hands and/or feet based on area of bother. An unpaired two tailed t-test statistical method (p<0.05) was used to analyze the significance of the mean difference between parameter variables after treatment and at baseline. Any response of N/A (not applicable) was not included in the analyses of the specific parameter.

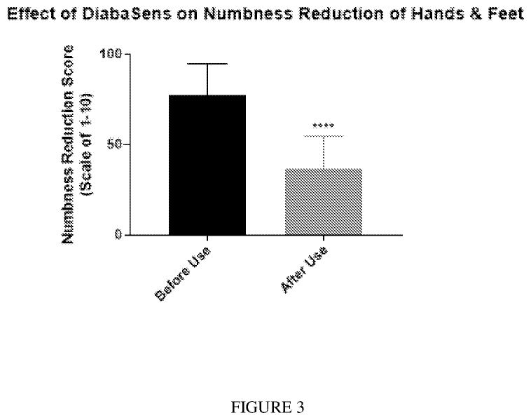

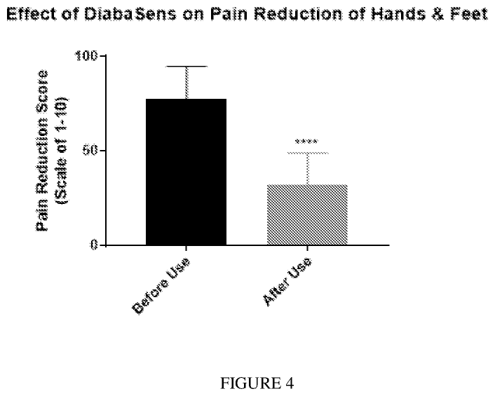

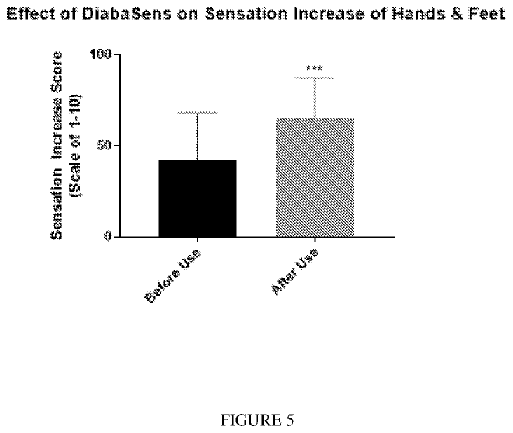

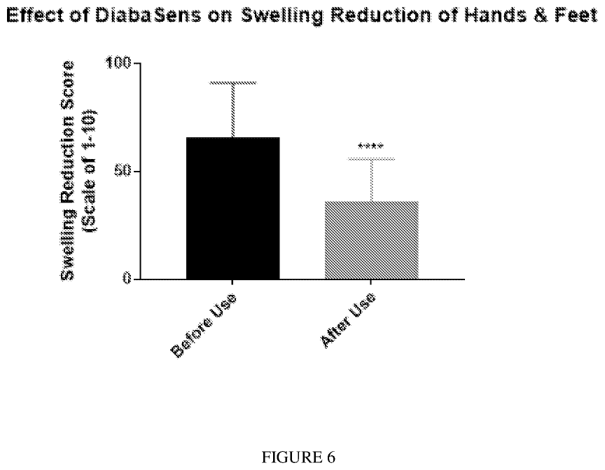

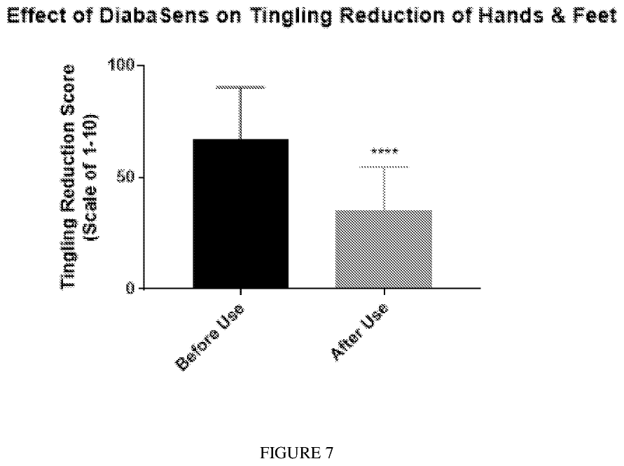

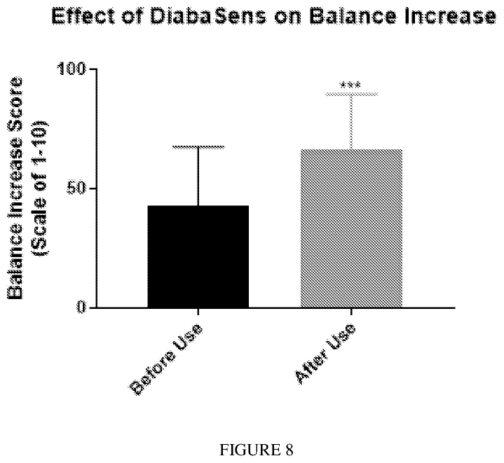

A total of 39 participants of which 15 had diabetes for an average of 13.5 years and 33 participants reported neuropathy. The average history of use was 1.5 weeks. The average reported onset effect following application was between 10-14.5 minutes. All measured parameters were statistically significant compared to baseline. Participants reported a reduction in pain by 41.7% (n=35), reduction in numbness 47.3% (n=35), increase in sensation 155.3% (n=31), reduced swelling 54.8% (n=27), reduced tingling 54.2% (n=28), and increase balance 155.4% (n=30) (FIGS. 3-8).

In a majority of study participants, formulations of Example 2 and Table 2 disclosed herein was superior to prior art products used products in terms of overall effectiveness. 34 out of the 39 responders (87.1%) observed an overall improvement with use. More than 84% (33 out of 39 participant) rated the instantly disclosed topical composition as the best product used. 100% of participants responded that they would recommend the formulation. The instantly disclosed topical formulation for human use is safe to use daily and effective in supporting healthy blood circulation while promoting sensation to the site of application.

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

What is claimed is:

1. A method for improving the sensitivity of the skin comprising applying to the skin an effective amount of a topical composition comprising sweet almond oil, cinnamon bark oil, coriander seed oil, L-arginine, and L-citrulline in an oil-in-water emulsion.

2. The method of claim 1, wherein the composition is applied to the skin.

3. The method of claim 2, wherein the skin is on the foot or hand.

4. The method of claim 3, wherein the skin is on the penis.

5. The method of claim 4, wherein the penis is circumcised or uncircumcised.

6. The method of any one of claims 1 to 5, wherein the composition comprises: about 6 wt. % sweet almond oil, about 0.75 wt. % cinnamon bark oil, about 0.5 wt. % coriander seed oil, about from about 0.33 wt. % to about 3.0 wt. % L-arginine, and from about 0.67 wt. % to about 6.0 wt. % L-citrulline.

7. The method of any one of claims 1 to 5, wherein the mass ratio of L-arginine to L-citrulline is about 1:2.

8. The method of any one of claims 1 to 5, wherein the mass ratio of sweet almond oil to cinnamon bark oil to coriander seed oil to L-arginine to L-citrulline is about 72:9:6:4:8.

9. The method of any one of claims 1 to 5, wherein the composition comprises: about 6 wt. % sweet almond oil, about 0.75 weight percent cinnamon bark oil, about 0.5 wt. % coriander seed oil, from about 0.34 to about 3.0 wt. % L-arginine, from about 0.68 to about 6.0 wt. % L-citrulline, about 5.0 wt. % of a 70 percent by weight sorbitol solution in water, about 4.0 wt. % of a 70 percent by weight isopropyl alcohol solution in water, about 20.0 wt. % propylene glycol, about 1.0 wt. % butylated hydroxytoluene, about 1.0 wt. % triethanolamine, about 1.0 wt. % benzyl alcohol, about 1.0 wt. % benzyl benzoate, about 3.5 wt. % PEG 40-hydrogenated castor oil, about 1.3 wt. % acrylate/C₁₀₋₃₀ alkyl acrylate crosspolymer, about 0.1 wt. % ethylenediaminetetraacetic acid disodium salt, and the balance water.

10. The method of any of claims 1 to 5, wherein a dose of about 0.5 mL to about 5 mL of the composition is applied to the skin from two to three times daily for a period of from two to three weeks, and from once to twice daily as needed thereafter.

11. A method for improving blood flow to the skin comprising applying to the skin an effective amount of a topical composition comprising sweet almond oil, cinnamon bark oil, coriander seed oil, L-arginine, and L-citrulline in an oil-in-water emulsion.

12. The method of claim 11, wherein the composition is applied to the skin.

13. The method of claim 12, wherein the skin is on the foot or hand.

14. The method of claim 11 wherein the skin is on the penis.

15. The method of claim 14, wherein the penis is circumcised or uncircumcised.

16. The method of any one of claims 11 to 15, wherein the composition comprises: about 6 wt. % sweet almond oil, about 0.75 wt. % cinnamon bark oil, about 0.5 wt. % coriander seed oil, from about 0.34 to about 3.0 wt. % L-arginine, and from about 0.68 to about 6.0 wt. % L-citrulline.

17. The method of any one of claims 11 to 15, wherein the mass ratio of L-arginine to L-citrulline is about 1:2.

18. The method of any one of claims 11 to 15, wherein the mass ratio of sweet almond oil to cinnamon bark oil to coriander seed oil to L-arginine to L-citrulline is about 72:9:6:4:8.

19. The method of any one of claims 11 to 15, wherein the composition comprises: about 6 wt. % sweet almond oil, about 0.75 weight percent cinnamon bark oil, about 0.5 wt. % coriander seed oil, from about 0.34 to about 3.0 wt. % L-arginine, from about 0.68 to about 6.0 wt. % L-citrulline, about 5.0 wt. % of a 70 percent by weight sorbitol solution in water, about 4.0 wt. % of a 70 percent by weight isopropyl alcohol solution in water, about 20.0 wt. % propylene glycol, about 1.0 wt. % butylated hydroxytoluene, about 1.0 wt. % triethanolamine, about 1.0 wt. % benzyl alcohol, about 1.0 wt. % benzyl benzoate, about 3.5 wt. % PEG 40-hydrogenated castor oil, about 1.3 wt. % acrylate/C₁₀₋₃₀ alkyl acrylate crosspolymer, about 0.1 wt. % ethylenediaminetetraacetic acid disodium salt, and the balance water.

20. The method of any of claims 11 to 15, wherein a dose of from about 0.5 mL to about 5 mL of the composition is applied to the skin from two to three times daily for a period of from two to three weeks and from once to twice daily as needed thereafter.

21. A method for treating neuropathy comprising applying to the skin an effective amount of a topical composition comprising sweet almond oil, cinnamon bark oil, coriander seed oil, L-arginine, and L-citrulline in an oil-in-water emulsion.

22. The method of claim 21, wherein the composition is applied to the skin.

23. The method of claim 22, wherein the skin is on the feet or hands.

24. Tis applied to the skin from two to three times daily for a period of from two to three weeks and from once to twice daily as needed thereafter.

25. The method of claim 24, wherein the penis is circumcised or uncircumcised.

26. The method of any one of claims 21 to 24, wherein the composition comprises: about 6 wt. % sweet almond oil, about 0.75 wt. % cinnamon bark oil, about 0.5 wt. % coriander seed oil, from about 0.34 to about 3.0 wt. % L-arginine, and from about 0.68 to about 6.0 wt. % L-citrulline.

27. The method of any one of claims 21 to 24, wherein the mass ratio of L-arginine to L-citrulline is about 1:2.

28. The method of any one of claims 21 to 24, wherein the mass ratio of sweet almond oil to cinnamon bark oil to coriander seed oil to L-arginine to L-citrulline is about 72:9:6:4:8.

29. The method of any one of claims 21 to 24, wherein the composition comprises: about 6 wt. % sweet almond oil, about 0.75 weight percent cinnamon bark oil, about 0.5 wt. % coriander seed oil, from about 0.34 wt. to about 3.0% L-arginine, from about 0.68 to about 6.0 wt. % L-citrulline, about 5.0 wt. % of a 70 percent by weight sorbitol solution in water, about 4.0 wt. % of a 70 percent by weight isopropyl alcohol solution in water, about 20.0 wt. % propylene glycol, about 1.0 wt. % butylated hydroxytoluene, about 1.0 wt. % triethanolamine, about 1.0 wt. % benzyl alcohol, about 1.0 wt. % benzyl benzoate, about 3.5 wt. % PEG 40-hydrogenated castor oil, about 1.3 wt. % acrylate/C₁₀₋₃₀ alkyl acrylate crosspolymer, about 0.1 wt. % ethylenediaminetetraacetic acid disodium salt, and the balance water.

30. The method of any of claims 21 to 29, wherein the dose of 0.5 to 5 mL of the composition are applied to the skin two to three times daily for a period of two to three weeks and once to twice daily as needed thereafter.

31. The method of any of claims 21 to 30, wherein the neuropathy is diabetic neuropathy.

32. A topical composition for improving blood flow to the skin and the sensitivity of the skin comprising sweet almond oil, cinnamon bark oil, coriander seed oil, L-arginine, and L-citrulline in an oil-in-water emulsion.

33. The composition of claim 33, wherein the composition comprises: about 6 wt. % sweet almond oil, about 0.75 wt. % cinnamon bark oil, about 0.5 wt. % coriander seed oil, from about 0.34 to about 3.0 wt. % L-arginine, and from about 0.68 to about 6.0 wt. % L-citrulline.

34. The composition of any one of claims 32 to 33, wherein the mass ratio of L-arginine to L-citrulline is approximately 1:2.

35. The composition of any one of claims 32 to 33, wherein the mass ratio of sweet almond oil to cinnamon bark oil to coriander seed oil to L-arginine to L-citrulline is about 72:9:6:4:8.

36. The composition of any one of claims 32 to 33, further comprising one or more pharmaceutically acceptable excipients.

37. The composition of any one of claims 32 to 33, wherein the composition comprises: about 6 wt. % sweet almond oil, about 0.75 weight percent cinnamon bark oil, about 0.5 wt. % coriander seed oil, from about 0.34 to about 3.0 wt. % L-arginine, from about 0.68 to about 6.0 wt. % L-citrulline, about 5.0 wt. % of a 70 percent by weight sorbitol solution in water, about 4.0 wt. % of a 70 percent by weight isopropyl alcohol solution in water, about 20.0 wt. % propylene glycol, about 1.0 wt. % butylated hydroxytoluene, about 1.0 wt. % triethanolamine, about 1.0 wt. % benzyl alcohol, about 1.0 wt. % benzyl benzoate, about 3.5 wt. % PEG 40-hydrogenated castor oil, about 1.3 wt. % acrylate/C₁₀₋₃₀ alkyl acrylate crosspolymer, about 0.1 wt. % ethylenediaminetetraacetic acid disodium salt, and the balance water.

38. The composition of any one of claims 32 to 33, wherein the composition comprises: about 6 wt. % sweet almond oil, about 0.75 weight percent cinnamon bark oil, about 0.5 wt. % coriander seed oil, from about 0.15 to about 1.0 wt. % L-arginine, from about 0.25 to about 2.0 wt. % L-citrulline, about 5.0 wt. % of a 70 percent by weight sorbitol solution in water, about 4.0 wt. % of a 70 percent by weight isopropyl alcohol solution in water, about 20.0 wt. % propylene glycol, about 1.0 wt. % butylated hydroxytoluene, about 1.0 wt. % triethanolamine, about 1.0 wt. % benzyl alcohol, about 1.0 wt. % benzyl benzoate, about 3.5 wt. % PEG 40-hydrogenated castor oil, about 1.3 wt. % acrylate/C₁₀₋₃₀ alkyl acrylate crosspolymer, about 0.1 wt. % ethylenediaminetetraacetic acid disodium salt, and the balance water.

39. The composition of any one of claims 26 to 27, wherein the composition comprises from about 150 mg to about 3.0 g L-arginine and from about 300 to about 3.0 g L-citrulline.
